(19) United States
(12) Patent Application Publication
(10) Pub. No.: US 2022/0089349 A1
(43) Pub. Date: Mar. 24, 2022

(54) Arrangement for a Flexible Bag

(71) Applicant: Global Life Sciences Solutions USA LLC, Marlborough, MA (US)

(72) Inventors: Christopher Prem Kumar, Bengaluru, Karnataka (IN); Manoj Kumar Ramakrishna, Bengaluru, Karnataka (IN)

(21) Appl. No.: 17/296,745

(22) PCT Filed: Mar. 19, 2019
(86) PCT No.: PCT/EP2019/056860

§ 371 Date: May 25, 2021

(30) Foreign Application Priority Data
Dec. 19, 2018 (EP) PCT/EP2018/085988

(51) Int. Cl. B65D 75/58

(52) U.S. Cl. CPC B65D 75/58; B65D 2588/545; B65D 2231/004

(57) ABSTRACT

A flexible bag (1; 101; 201) comprising a component (3; 103; 203) inside the bag, wherein said flexible bag comprises a receiving device (5; 105; 205) provided in a wall (7; 107; 207) of the flexible bag, wherein said component (3; 103; 203) and said receiving device (5; 105; 205) are configured for being connectable to each other for keeping the component substantially stationary within the flexible bag when so connected.

### TECHNICAL FIELD OF THE INVENTION

The present invention relates to a flexible bag and to a component positioned inside a flexible bag and to a method for handling a flexible bag comprising a component inside the flexible bag.

### BACKGROUND OF THE INVENTION

When assembling, folding and transporting flexible bags there may be problems if harder components of the flexible bag are pressed against the flexible walls of the bag. There is a risk that dents are formed in the flexible walls or even ruptures. For example if a perfusion filter is provided in a flexible bioreactor bag, also called a cellbag, said perfusion filter can be connected to a waste outlet via an elbow connector, i.e. a connector comprising a 90° bend. When the bag is empty, i.e. before use, for example during assembly of the bag and during transport and storage of the bag there is a risk that the elbow connector is pressed against the flexible walls of the bag. This may cause dents or ruptures or deformation to the walls of the bag.

### SUMMARY

An object of the invention is to provide a flexible bag, which flexible bag comprises a component inside the flexible bag and which flexible bag can be assembled, stored and transported with reduced risk that the flexible bag is damaged.

A further object of the invention is to provide a component which can be used inside a flexible bag with reduced risk of providing damage to the flexible bag.

This is achieved by a flexible bag, a component and a method according to the independent claims.

According to one aspect of the invention a flexible bag is provided comprising a component inside the bag, wherein said flexible bag comprises a receiving device provided in a wall of the flexible bag, wherein said component and said receiving device are configured for being connectable to each other for keeping the component substantially stationary within the flexible bag when so connected.

Hereby a flexible bag comprising a component inside the bag is provided which can be assembled, transported and stored with reduced risk that the component will damage the flexible walls of the bag.

According to another aspect of the invention a component is provided configured for being positioned inside a flexible bag, wherein said component comprises a locating part configured such that it can be connected to a receiving device provided in the flexible bag such that the component is kept in a wanted position within the flexible bag.

Hereby a component is provided which can be provided inside a flexible bag and which component can be secured within a receiving device of the flexible bag such that the component does not risk to damage the flexible walls of the bag during assembly of the bag, during transport of the bag or during storage of the bag.

According to another aspect of the invention a method is provided for handling a flexible bag comprising a component inside the flexible bag, said method comprising the steps of:
providing a component inside a flexible bag;
providing a receiving device in a wall of the flexible bag;
connecting the component with the receiving device during assembly and/or transport and/or storage and/or use of the flexible bag;
releasing the component from the receiving device during assembly and/or transport and/or storage and/or use of the flexible bag.

Hereby a method is provided for connecting a component provided inside a flexible bag such that it is secured in a wanted position inside the flexible bag during some parts of a flexible bag handling process, such as for example during transport and storage of the flexible bag. Furthermore the component can be released during another part of the flexible bag handling process, such as for example during use of the flexible bag. If the flexible bag for example is a bioreactor, use of the flexible bag can be cell cultivation. If the component is a fluid connector of a perfusion filter the component should be released during use. If instead the component is an impeller system the component should be connected to the receiving device during use in order to stabilize the impeller system inside the flexible bag. With the method according to the invention there is less risk that the component provide damage to the flexible bag because the component can be kept in a wanted position in relation to the walls of the flexible bag.

Embodiments of the invention are described in the dependent claims.

### BRIEF DESCRIPTION OF THE DRAWINGS

FIG. 1 is a cross section of a flexible bag and a component provided inside the flexible bag according to one embodiment of the invention.

FIG. 2 is a perspective view of a component with a locating part according to one embodiment of the invention.

FIG. 3 is a cross section of a flexible bag and a component provided inside the flexible bag according to one embodiment of the invention.

FIG. 4 is a perspective view of a component with a locating part according to one embodiment of the invention.

FIG. 5 is a perspective view of a flexible bag comprising an impeller system.

### DETAILED DESCRIPTION OF THE EMBODIMENTS

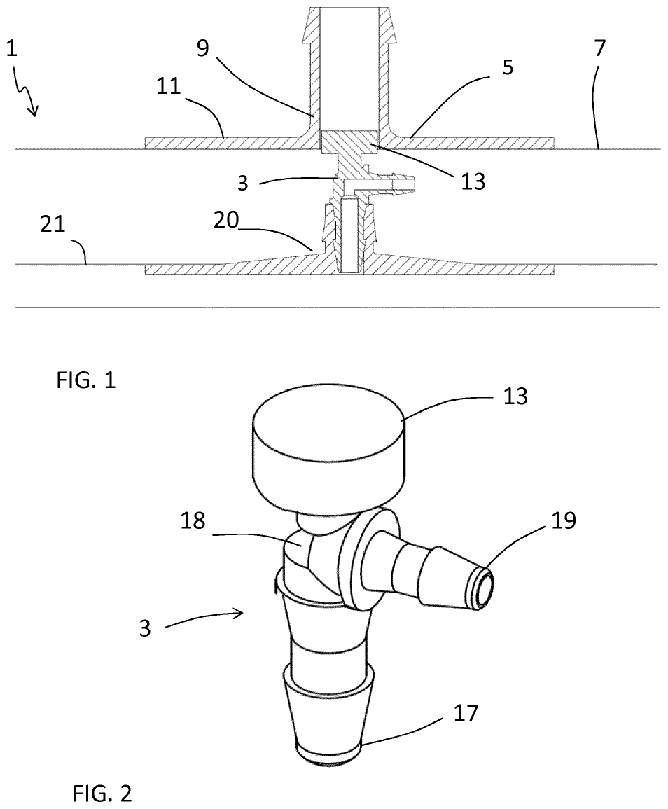

FIG. 1 is a cross section of a flexible bag 1 and a component 3 provided inside the flexible bag according to one embodiment of the invention. According to the invention the flexible bag 1 comprises a receiving device 5 provided in a wall 7 of the flexible bag and the component 3 and said receiving device 5 are configured for being connectable to each other for keeping the component substantially stationary within the flexible bag when so connected. The component 3 and the receiving device 5 are configured for being releasably connectable to each other. In the embodiment shown in FIG. 1 the component 3 is an elbow fluid connector which is connected to a port 20 of a perfusion filter 21 provided inside the flexible bag 1. The flexible bag is in this example a bioreactor. The receiving device 5 is in this embodiment a port provided in a flexible wall 7 of the flexible bag. This could be one of the ports which is used for fluid transfer in and out of the flexible bag during use of the flexible bag. The receiving device 5 comprises in this embodiment of the invention a connection part 9 for connection to the component 3 and a bag attaching part 11 which is configured such that it can be attached to a wall 7 of the flexible bag 1 and configured for protecting the walls of the flexible bag from the component. I.e. the bag attaching part 11 is made more robust than the walls of the flexible bag and it is made from a material which can be sealed to the walls of the flexible bag. The receiving device can be injection molded from a plastic material.

FIG. 2 is a perspective view of the component 3 as used in FIG. 1, i.e. an elbow fluid connector. In this embodiment of the invention the component 3 comprises a locating part 13. Said locating part 13 and said receiving device 5 of the flexible bag 1 are configured for being releasably connectable to each other for keeping the component in a wanted position within the flexible bag 1. The locating part 13 comprises here a cylindrical head which can be inserted into the receiving device 5 which is a port. The locating part 13 is provided extending from the elbow fluid connector in a direction being the same direction as either an inlet port 17 or an outlet port 19 of the elbow fluid connector is extending. The locating part 13 is in this embodiment of the invention provided to an end of the elbow fluid connector which is opposite an inlet port 17 and it is attached to a bend 18 of the elbow fluid connector where the fluid flow is changing direction inside the elbow fluid connector.

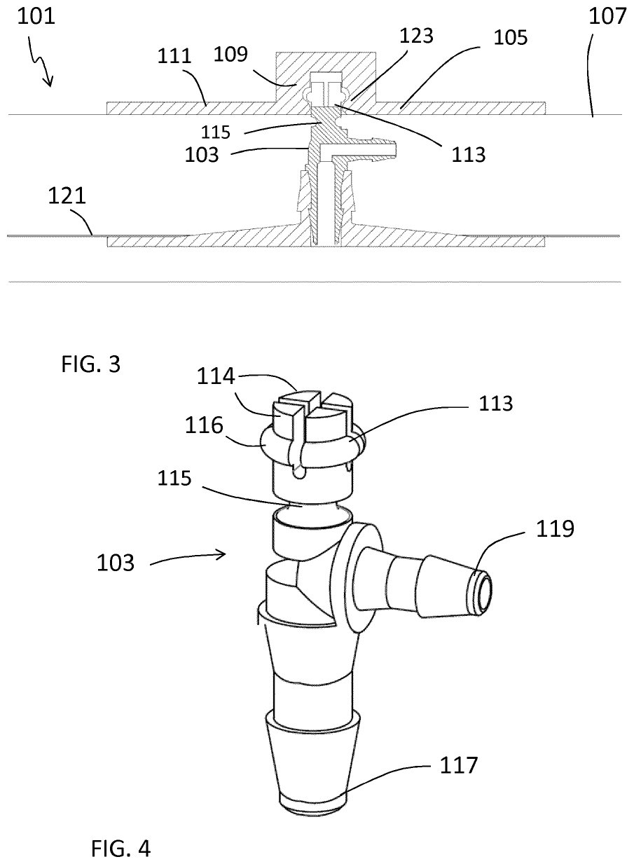

FIG. 3 is a cross section of a flexible bag 101 and a component 103 provided inside the flexible bag according to another embodiment of the invention. FIG. 4 is a perspective view of the component 103 provided in the flexible bag 101 of FIG. 3. The component 103 is also in this embodiment an elbow fluid connector provided with a locating part 113. In FIG. 3 the elbow fluid connector 103 is shown connected to a perfusion filter 121 which is provided inside the flexible bag 101. The flexible bag 101 comprises a receiving device 105 which in this example is a dummy port provided to a flexible wall 107 of the flexible bag 101. The difference from the embodiment of the component 3 shown in FIG. 2 is that the locating part 113 of the component 103 in this embodiment comprises a compressible head which can be compressed for snap fitting inside the receiving device 105 and said head is provided on a breakable neck 115 which will break and thereby separate the head from the rest of the component when exposed to a predefined breaking force. Furthermore the receiving device 105 of the flexible bag 101 comprises a snap receiving feature 123. Hereby the component 103 can be snap fitted into the receiving device 105 when the component 103 needs to be kept in a specific position with the flexible bag 101. This could be for example during transport or storage of the flexible bag. When the flexible bag later should be used and the perfusion filter 121 needs to be provided free floating inside the flexible bag a force can be applied to break the breakable neck 115 and hereby release the head from the rest of the component 103. The compressible head of the locating part 113 can comprise resilient parts 114 and a snap fitting feature 116 which can fit inside a corresponding snap receiving feature 123 of the receiving device 105.

In another embodiment of the invention the receiving device can comprise a clip which can be controlled from outside the flexible bag to clip around the component for holding the component in a specific position and to release the component. Hereby no locating part needs to be provided to the component. In another embodiment the connection between the component and the receiving device could be accomplished by magnetic force, whereby either the receiving device or the component comprises a magnet and the other comprises a metallic material. In that case the magnet and metallic material are suitably over molded or protected by any other manufacturing techniques so as not to come in direct contact with the fluid used inside the bag.

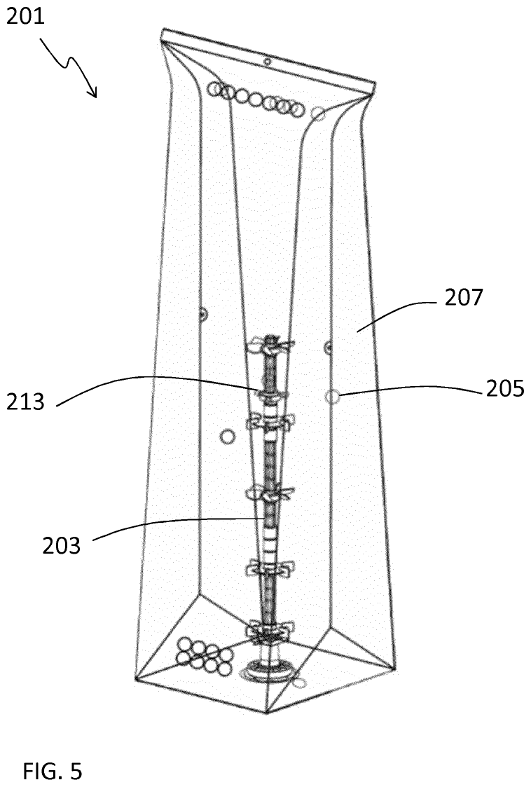

FIG. 5 is a perspective view of a flexible bag 201 according to another embodiment of the invention. The component 203 provided inside the flexible bag 201 is in this embodiment an impeller system. The impeller system is a hard component and there is a risk that the walls 207 of the flexible bag 201 can be damaged by the impeller system 203. According to the invention a receiving device 205 (not shown in detail in FIG. 5—number 205 is only pointing to a suitable position for such a receiving device which could be designed similar as discussed above) is provided in a wall 207 of the flexible bag 201 and the component 203, i.e. the impeller system, and said receiving device 205 are configured for being connectable to each other for keeping the impeller system in a wanted position within the flexible bag. In this embodiment of the invention the component 203 needs to be kept in correct position within the flexible bag during use of the system. One or more locating parts 213 (not shown in FIG. 5 but number 213 is pointing to a suitable position for such locating parts which can be designed similar as discussed above) can be provided to for example a bearing provided to an impeller shaft of the impeller system 203. Said locating parts 213 can be connected to one or more receiving devices 205 provided in the walls of the flexible bag 201.

According to the invention a method for handling a flexible bag 1; 101; 201 comprising a component 3; 103; 203 inside the flexible bag is also provided. Said method comprises the steps of:
providing a component 3; 103; 203)inside a flexible bag 1; 101; 201;
providing a receiving device 5; 105, 205 in a wall 7; 107; 207 of the flexible bag 1; 101; 201;
connecting the component 3; 103; 203 with the receiving device 5; 105; 205 during assembly and/or transport and/or storage and/or use of the flexible bag;
releasing the component 3; 103; 203 from the receiving device 5; 105; 205 during assembly and/or transport and/or storage and/or use of the flexible bag.

For the embodiments of the invention as shown in FIGS. 1 and 3, where a perfusion filter inside a flexible bioreactor bag is described the component 3; 103 need to be connected to the receiving device 5; 105 during transport and/or storage and needs to be released during use of the flexible bag. For the embodiment as shown in FIG. 5 where the component 203 is an impeller system the component needs to be connected during use of the flexible bag.

For the embodiment where the component 3; 103 is an elbow fluid connector comprising a locating part 13; 113 the step of providing the component inside the flexible bag further comprises providing the elbow fluid connector 3; 103 to a perfusion filter 21; 121 inside the flexible bag 1; 101 and the step of connecting the component 3; 103 to the receiving device 5; 105 comprises connecting the locating part 13; 113 of the elbow fluid connector to the receiving device 5; 105 during assembly and/or transport and/or storage of the flexible bag and the step of releasing the component 3; 103 from the receiving device 5; 105 is performed during use of the flexible bag. The step of connecting the component 3; 103 with the receiving device 5; 105 comprises inserting said cylindrically formed head into said receiving device.

For the embodiment as shown in FIG. 4 where the locating part 113 comprises a cylindrically formed head comprising resilient parts and a breakable neck 115, the method step of releasing the component 103 from the receiving device 105 comprises breaking said neck 115 thus separating said head from the rest of the component.

Other examples of components which can be provided inside a flexible bag and connected to a receiving device provided to a wall of the flexible bag according to the invention are sensors or sensor casings, other types of impellors, sparging components or tubing.

1. A flexible bag comprising a component inside the bag, wherein said flexible bag comprises a receiving device provided in a wall of the flexible bag, wherein said component and said receiving device are configured for being connectable to each other for keeping the component substantially stationary within the flexible bag when so connected.

2. The flexible bag according to claim 1, wherein said component and said receiving device are configured for being releasably connectable to each other.

3. The flexible bag according to claim 1, wherein said receiving device comprises a connection part connection to the components and a bag attaching part which is configured such that it can be attached to a wall of the flexible bag and configured for protecting the walls of the flexible bag from the component.

4. The flexible bag according to claim 1, wherein said component comprises a locating part and wherein said locating part and said receiving device are configured for being connectable to each other for keeping the component in a wanted position within the flexible bag.

5. The flexible bag according to claim 4, wherein said locating part comprises a cylindrical head and said receiving device comprises a port into which said locating part can be inserted.

6. The flexible bag according to claim 4, wherein said locating part comprises a compressible head which can be compressed for snap fitting inside the receiving device and wherein said head is provided on a breakable neck which will break and thereby separate the head from the rest of the component when exposed to a predefined breaking force.

7. The flexible bag according to claim 4, wherein said component is an elbow fluid connector and wherein said locating part is provided extending from the elbow fluid connector in a direction being the same direction as either an inlet port or an outlet port of the elbow fluid connector is extending.

8. The flexible bag according to claim 7, wherein the flexible bag comprises a perfusion filter and the elbow fluid connector connected to the perfusion filter.

9. The flexible bag according to claim 1, wherein the flexible bag is a bioreactor bag.

10. The flexible bag according to claim 1, wherein the receiving device is a port provided in a wall of the flexible bag.

11. The flexible bag according to claim 1, wherein the receiving device is a dummy port comprising snap receiving features.

12. The flexible bag according to claim 1, wherein the receiving device comprises a clip which can be controlled from outside the flexible bag to clip around the component and to release the component.

13. The flexible bag according to claim 1, wherein the connection between the component and the receiving device is accomplished by magnetic force, whereby either the receiving device or the component comprises a magnet and the other comprises a metallic material.

14. The flexible bag according to claim 1, wherein the component is an impeller system.

15. A component configured for being positioned inside a flexible bag, wherein said component comprises a locating part configured such that it can be connected to a receiving device provided in the flexible bag such that the component is kept in a wanted position within the flexible bag.

16. The component according to claim 15, wherein said component is a bioreactor internal component.

17. The component according to claim 15, wherein said component is an elbow fluid connector and wherein said locating part is provided extending from the connector in a direction being the same direction as either an inlet port or an outlet port of the connector is extending.

18. The component according to claim 15, wherein said locating part comprises a cylindrically formed head.

19. The component according to claim 18, wherein said head comprises resilient parts such that the head can be compressed for snap fitting inside the receiving device of the flexible bag and wherein said head is provided on a breakable neck which will break and thereby separate the head from the rest of the component when exposed to a predefined breaking force.

20. A method for handling a flexible bag comprising a component inside the flexible bag, said method comprising the steps of:
providing a component inside a flexible bag
providing a receiving device in a wall of the flexible bag;
connecting the component with the receiving device during assembly and/or transport and/or storage and/or use of the flexible bag; and
releasing the component from the receiving device during assembly and/or transport and/or storage and/or use of the flexible bag.

21. The method for handling a flexible bag according to claim 20, wherein the component is an elbow fluid connector comprising a locating part and the step of providing the component inside the flexible bag comprises further providing the elbow fluid connector to a perfusion filter inside the flexible bag and the step of connecting the component to the receiving device comprises connecting the locating part the elbow fluid connector to the receiving device during assembly and/or transport and/or storage of the flexible bag and the step of releasing the component from the receiving devices is performed during use of the flexible bag.

22. A The method according to claim 21, wherein the locating part comprises a cylindrically formed head and wherein the step of connecting the component with the receiving device comprises inserting said cylindrically formed head into said receiving device.

23. A The method according to claim 21, wherein the locating part comprises a cylindrically formed head, wherein said head comprises resilient parts such that the head can be compressed for snap fitting inside the receiving device of the flexible bag and wherein said head is provided on a breakable neck which will break and thereby separate the head from the rest of the component when exposed to a predefined breaking force, wherein said step of releasing the component from the receiving device comprises breaking said neck thus separating said head from the rest of the component.

24. The method for handling a flexible bag according to claim 20, wherein the component is an impeller system and the step of connecting the component to the receiving device is performed during use of the flexible bag.
